(19) United States
(12) Patent Application Publication
(10) Pub. No.: US 2016/0262801 A1
(43) Pub. Date: Sep. 15, 2016

(54) SPINAL IMPLANT SYSTEM AND METHODS OF USE

(71) Applicant: Warsaw Orthopedic, Inc., Warsaw, IN (US)

(72) Inventors: William Alan Rezach, Atoka, TN (US); Jason M. May, Cordova, TN (US); Gary Lindemann, Collierville, TN (US)

(21) Appl. No.: 14/656,386

(22) Filed: Mar. 12, 2015

(51) Int. Cl. A61B 17/70

(52) U.S. Cl. CPC A61B 17/7032; A61B 17/7037

(57) ABSTRACT

A spinal implant system comprises a plurality of alternate first members including at least one first member. The first member comprises an inner surface defining an implant cavity, and a first part being non-rotatable relative to the inner surface and a second part movable relative to the first part. A second member includes a mating element engageable with a first member such that the second member is interchangeable with the plurality of first members. The first member is rotatable relative to the second member in a first plane of a body and the second part defines a portion of the implant cavity and is movable relative to the first part in a second plane of the body. Fasteners, instruments and methods are disclosed.

### TECHNICAL FIELD

The present disclosure generally relates to medical devices for the treatment of spinal disorders, and more particularly to a surgical implant system including a bone fastener.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, spinal constructs such as vertebral rods are often used to provide stability to a treated region. Rods redirect stresses away from a damaged or defective region while healing takes place to restore proper alignment and generally support the vertebral members. During surgical treatment, one or more rods and bone fasteners can be delivered to a surgical site. The rods may be attached via the fasteners to the exterior of two or more vertebral members. This disclosure describes an improvement over these prior art technologies.

### SUMMARY

In one embodiment, a spinal implant system is provided. The spinal implant system comprises a plurality of alternate first members including at least one first member. The at least one first member comprises an inner surface defining an implant cavity. The at least one first member includes a first part being non-rotatable relative to the inner surface and a second part movable relative to the first part. A second member is configured to penetrate tissue and includes a mating element engageable with a first member such that the second member is interchangeable with the plurality of first members. The at least one first member is rotatable relative to the second member in a first plane of a body and the second part defines a portion of the implant cavity and is movable relative to the first part in a second plane of the body. In some embodiments, fasteners, instruments and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a spinal implant system in accordance with the principles of the present disclosure;

FIG. 2 is a perspective view of components of the system shown in FIG. 1;

FIG. 3 is a plan view of the components shown in FIG. 2;

FIG. 4 is a plan view of components of the system shown in FIG. 1;

FIG. 5 is a perspective view of components of the system shown in FIG. 1;

FIG. 6 is a break away view of the components shown in FIG. 5;

FIG. 7 is a perspective view of the components shown in FIG. 1 with parts separated;

FIG. 8 is a perspective view of the components shown in FIG. 1 with parts separated;

FIG. 9 is a cross section view of the components shown in FIG. 1;

FIG. 10 is a perspective view of the components shown in FIG. 1;

FIG. 11 is a cross section view of the components shown in FIG. 1;

FIG. 12 is a cross section view of the components shown in FIG. 1;

FIG. 13 is a perspective view of components of one embodiment of a spinal implant system in accordance with the principles of the present disclosure;

FIG. 14 is a perspective view of components of one embodiment of a spinal implant system in accordance with the principles of the present disclosure;

FIG. 15 is a perspective view of components of the system shown in FIG. 14;

FIG. 16 is a perspective view of components of the system shown in FIG. 14; and

FIG. 17 is a perspective view of the components shown in FIG. 14 in part phantom.

### DETAILED DESCRIPTION

The exemplary embodiments of a surgical system and related methods of use disclosed are discussed in terms of medical devices for the treatment of musculoskeletal disorders and more particularly, in terms of a spinal implant system including a bone fastener. In one embodiment, the spinal implant system includes an implant comprising a bone fastener.

In some embodiments, the spinal implant system comprises a selectively coupled transverse, sagittal adjusting receiver. In some embodiments, the spinal implant system includes a receiver that is configured to accommodate transverse and sagittal anatomical differences. In some embodiments, the spinal implant system comprises a modular system including an array of members, such as, for example, receivers that are selectively coupled to members, such as, for example, bone screw shafts. In some embodiments, the spinal implant system facilitates sagittal correction and/or manipulation when a spinal rod is disposed with a receiver.

In some embodiments, the spinal implant system comprises a bone fastener having a universal screw design that includes one or more flats and/or a keyed geometry to facilitate transverse angulation. In some embodiments, the bone fastener includes a screw shank having two flats on an outside surface thereof.

In some embodiments, the spinal implant system comprises a bone fastener including a head, a saddle, a crown, pins that retain the saddle with the crown and a ring that retains the head with a screw shaft. In some embodiments, the spinal implant system comprises a bone fastener including a head, a crown, a saddle having protrusions that mate within channels of the crown, and a ring that retains the head with a screw shaft. In some embodiments, the protusions of the saddle engage the head at a full pivot limit of the components.

In some embodiments, the spinal implant system comprises a bone fastener including a saddle that fits within a crown outer profile. In some embodiments, the saddle is inserted upwardly through the head. In some embodiments, the spinal implant system includes at least one pin configured for insertion and retained within a pocket disposed with the receiver when assembled. In some embodiments, pins are inserted from exterior to the receiver and retained by a pocket in the receiver once the sub-assembly of components is inserted into the receiver. In some embodiments, the spinal implant system includes a bone fastener having a smaller receiver profile.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed spinal implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic, sacral and pelvic regions of a spinal column. The spinal implant system of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

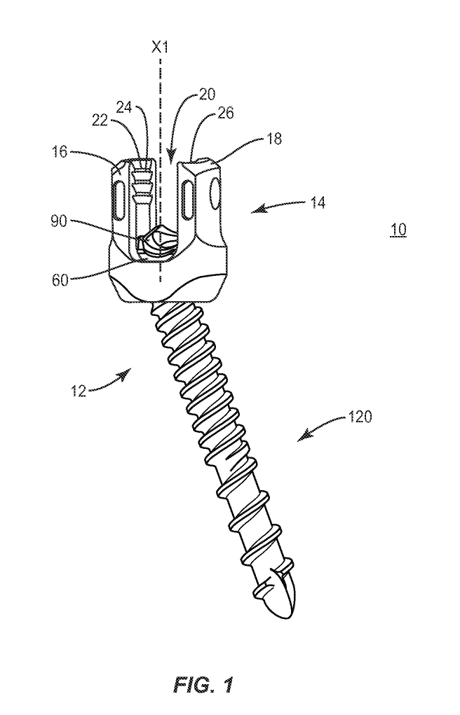

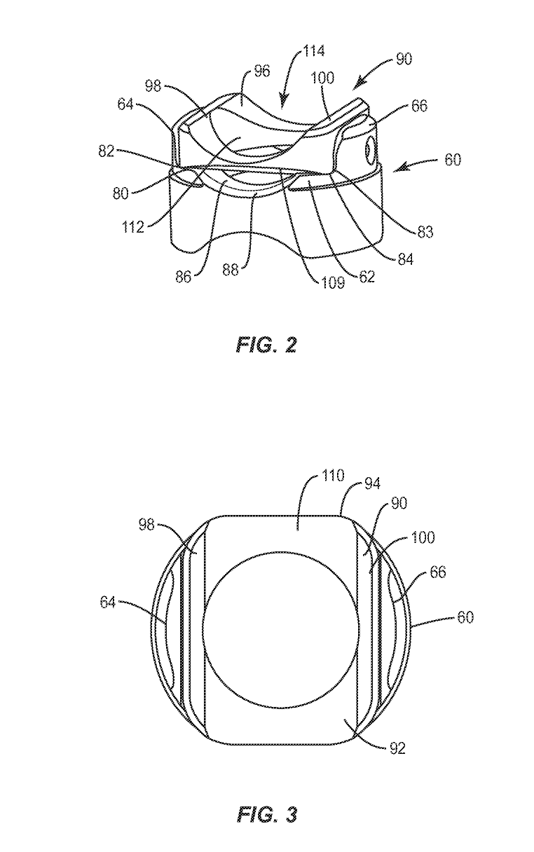

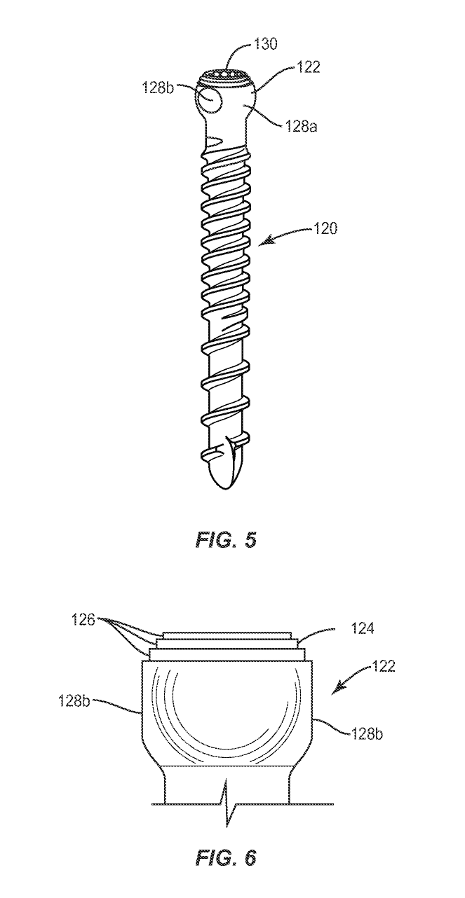

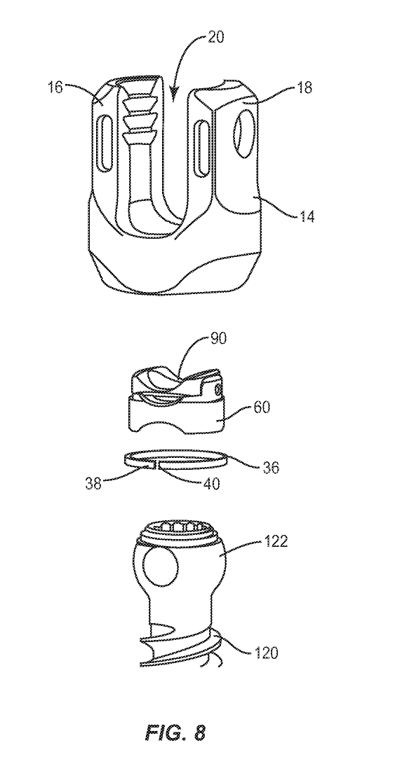

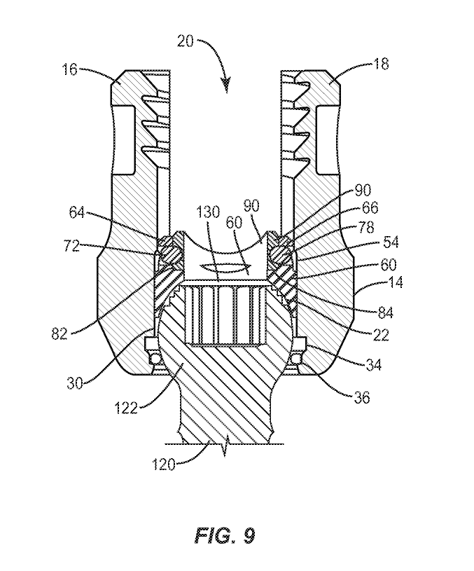

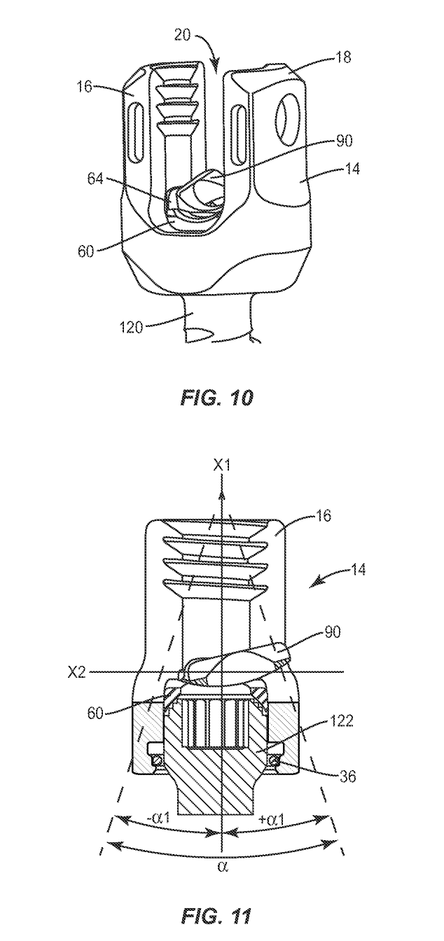

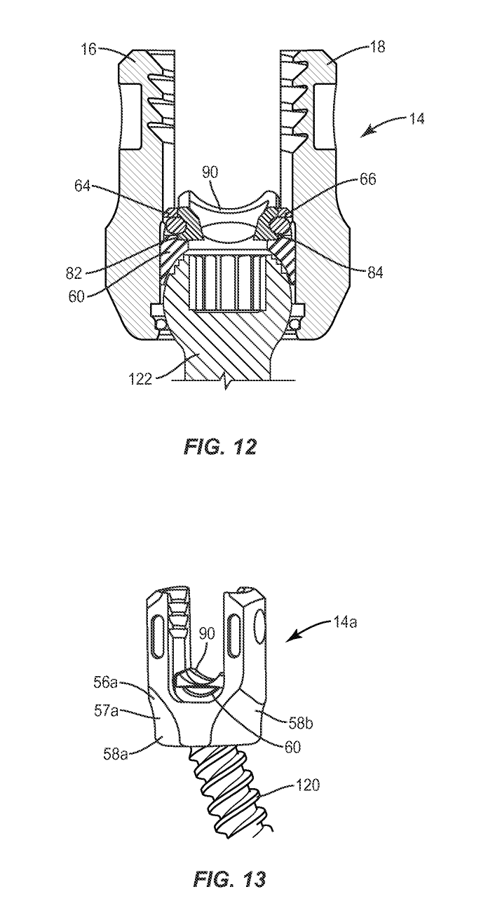

The following discussion includes a description of a surgical system including a bone fastener, related components and methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-12, there are illustrated components of a spinal implant system 10 including a plurality of alternate bone fastener configurations, such as, for example, a plurality of bone screw configurations 12.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherirmide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TOP), hydroxyapatite (HA)-TCP, calcium sulfate, or other resorbable polymers such as polyaetide, polyglycolide, polytyrosine carbonate, polycaroplaetohe and their combinations.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

In some embodiments, spinal implant system 10 comprises a spinal implant kit, which includes a plurality of members, such as, for example, implant receivers 14. Receiver 14 is configured for selection from the plurality of receivers such that receiver 14 is connectable with an interchangeable member, such as, for example, a shaft 120. In some embodiments, receiver 14 is configured for selection from the plurality of receivers such that receiver 14 is connectable with a compatible shaft 120.

An interchangeable mating element, such as, for example, a head 122 of shaft 120 is interchangeable with a mating element, as described herein, of each of the plurality of receivers 14 to form a selected bone screw 12 having a selected movement of its components parts and/or movement relative to tissue. In some embodiments, the selected movement includes rotation and/or pivotal movement of shaft 120 relative to receiver 14 about one axis. In some embodiments, the selected movement includes rotation and/or pivotal movement of shaft 120 relative to receiver 14 through one plane. In some embodiments, shaft 120 is connected to a selected receiver 14 to comprise a uni-axial fastener. In some embodiments, spinal implant system 10 comprises a spinal implant kit, which includes receivers 14 and alternate receivers, such as those described herein.

Each receiver 14 extends along and defines an axis X1. Each receiver 14 includes a pair of spaced apart arms 16, 18 that define an implant cavity 20 therebetween configured for disposal of a component of a spinal construct, such as, for example, a spinal rod (not shown). Arms 16, 18 each extend parallel to axis X1, as shown in FIG. 1. In some embodiments, arm 16 and/or arm 18 may be disposed at alternate orientations, relative to axis X1, such as, for example, transverse, perpendicular and/or other angular orientations such as acute or obtuse, coaxial and/or may be offset or staggered. Arms 16, 18 each include an arcuate outer surface extending between a pair of side surfaces. At least one of the outer surfaces and the side surfaces of arms 16, 18 have at least one recess or cavity therein configured to receive an insertion tool, compression instrument and/or instruments for inserting and tensioning bone screw 12. In some embodiments, arms 16, 18 are connected at proximal and distal ends thereof such that receiver 14 defines a dosed spinal rod slot.

Cavity 20 is substantially U-shaped. In some embodiments, all or only a portion of cavity 20 may have alternate cross section configurations, such as, for example, dosed, V-shaped, W-shaped, oval, oblong triangular, square, polygonal, irregular, uniform, non-uniform, offset, staggered, and/or tapered. Each receiver 14 includes an inner surface 22. A portion of surface 22 includes a thread form 24 located adjacent arm 16 and a thread form 26 located adjacent arm 18. Thread forms 24, 26 are each configured for engagement with a coupling member, such as, for example, a setscrew (not shown), to retain a spinal construct, such as, for example, a spinal rod (not shown) within cavity 20. In some embodiments, surface 22 may be disposed with the coupling member in alternate fixation configurations, such as, for example, friction fit, pressure fit, locking protrusion/recess, locking keyway and/or adhesive. In some embodiments, all or only a portion of surface 22 may have alternate surface configurations to enhance engagement with the spinal rod and/or the setscrew such as, for example, rough, arcuate, undulating, mesh, porous, semi-porous, dimpled and/or textured.

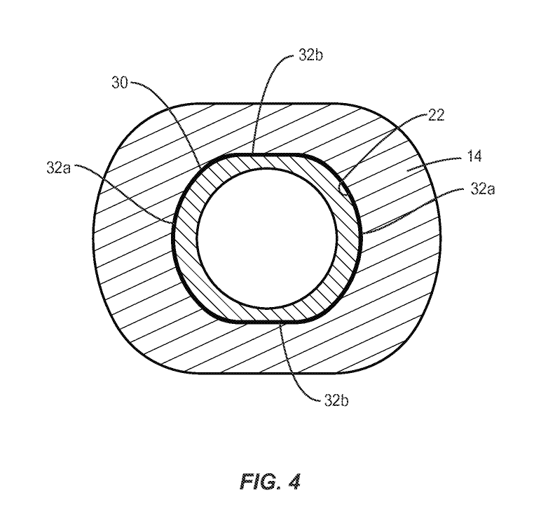

A portion of surface 22 of each receiver 14 defines a particularly configured mating element, such as, for example, an engagement surface 30 configured to interface in a selective mating engagement with head 122 of shaft 120, as shown in FIG. 4. Surface 30 includes a keyway 32 that includes mating elements, such as, for example, arcuate surfaces 32a and planar surfaces, such as, for example, flats 32b.

Flats 32b are configured to interface with flats 128b of head 122 and arcuate surfaces 32a are configured to interface with arcuate surfaces 128a in a keyed connection. Flats 32b engage flats 128b to resist and/or prevent rotation of receiver 14 about axis X1. In this configuration, shaft 120 is free to rotate along a single axis and/or within a single plane relative to receiver 14. Head 122 is engageable with surface 30 and movable relative thereto such that shaft 120 is rotatable within a single plane, such as, for example, a transverse plane of a body and/or vertebrae relative to receiver 14. Interchangeable shaft 120 is connected with a selected receiver 14 from the kit of receivers 14. In some embodiments, receiver 14 may be disposed with shaft 120 in alternate fixation configurations, such as, for example, friction fit, pressure fit, locking protrusion/recess, locking keyway and/or adhesive.

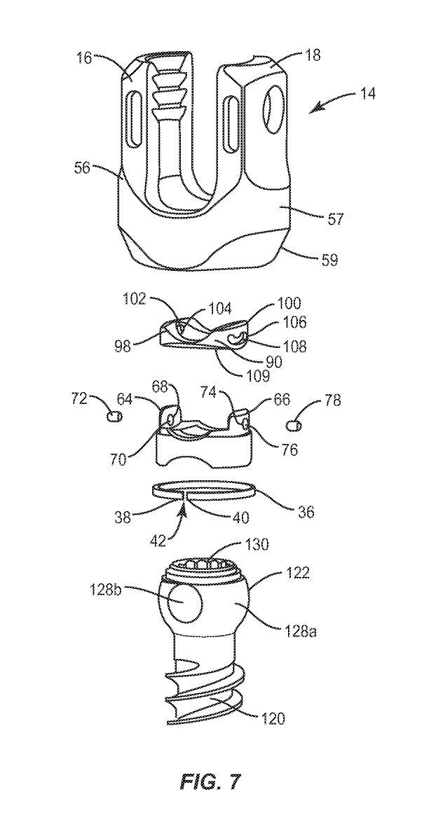

Each surface 22 defines a cavity, such as, for example, a groove 34 configured for disposal of a band, such as, for example, a C-shaped ring 36. Ring 36 includes a circumference that extends between end 38 and end 40. Ends 38, 40 define an opening, such as, for example a gap 42, as shown in FIG. 7. In some embodiments, gap 42 is sized such that gap 42 has a thickness that is less than a height and a width of ring 36. Ring 36 is configured to engage an outer surface of head 122 and is disposable with groove 34 to resist and/or prevent axial translation of shaft 120 relative to the selected receiver 14 and facilitate rotation of shaft 120 relative a selected receiver 14. In some embodiments, ring 36 is disposed within head 122 to enhance a retaining strength of bone screw 12 and resist and/or prevent shearing of shaft 120. In some embodiments, each surface 22 includes a cavity, such as, for example, a slot 54 configured to receive a flange of a part, such as, for example, a crown 60, as discussed herein.

Receiver 14 includes a surface 56 having an enlarged and/or oblong profile and/or perimeter 57 adjacent distal portions of arms 16, 18 and a tapered portion 59 disposed at a distal portion of receiver 14, as shown in FIG. 7. In some embodiments, as shown in FIG. 13, a receiver 14a, similar to receiver 14, includes a surface 56a having a substantially uniform profile and/or perimeter 57a. Surface 56a includes tapered portions 58a, 58b.

Surface 22 includes an inner profile that defines a perimeter of cavity 20. Crown 60 is configured for disposal within the inner profile of surface 22 and/or the perimeter of cavity 20 of the selected receiver 14. Crown 60 includes an outer profile and/or perimeter that fits within the inner profile of surface 22. An outer surface of crown 60 engages surface 22. In some embodiments, arms 64, 66, described herein, are keyed to a portion of surface 22 defining slot 54. In some embodiments, arms 64, 66 engage surface 22 such that crown 60 is fixed in rotation with surface 22 and/or non-rotatable about axis X1. In some embodiments, arms 64, 66 engage surface 22 such that crown 60 is fixed in rotation with surface 22 and/or non-rotatable about axis X1, and arms 64, 66 are translatable within slot 54 such that crown 60 is translatable relative to surface 22 and along axis X1. In some embodiments, this configuration facilitates translation of crown 60 within slot 54, which facilitates positioning of head 122 with a selected receiver 14 so that head 122 can be locked with a selected receiver 14, as described herein.

Crown 60 includes a wall 62 defining an extension, such as, for example, an arm 64 and an extension, such as, for example, an arm 66. Arms 64, 66 are configured to support relative movement of a part, such as, for example, a saddle 90. Arm 64 includes a surface 68 that defines an opening 70 configured for disposal of a pin 72, as described herein. Arm 66 includes a surface 74 that defines an opening 76 configured for disposal of a pin 78, as described herein. Pins 72, 78 are configured to facilitate connection of saddle 90 with crown 60 and relative motion thereof.

Wall 62 includes a surface 80 that defines a track 82 adjacent arm 64. Wall 62 includes a surface 83 that defines a track 84 adjacent arm 66. Tracks 82, 84 are configured to facilitate translation of saddle 90 relative to crown 60, as described herein. Arms 64, 66 are configured to guide saddle 90 along tracks 82, 84 relative to crown 60. Wall 62 includes a surface 86 that defines an arcuate portion 88 configured for disposal of at least a portion of an implant, such as, for example, a spinal rod (not shown), which may be positioned with fastener 12 and/or vertebral tissue.

Saddle 90 extends between an end 92 and an end 94. Saddle 90 includes a surface 96 defining a wall 98 and a wall 100. Walls 98, 100 are configured to fit within the outer profile and/or perimeter of crown 60. In some embodiments, saddle 90 fits within the outer profile and/or perimeter of crown 60 such that the sub-assembly of crown 60/saddle 90 is disposed, such as, for example, inserted and/or loaded upwardly through a lower opening of receiver 14 that communicates with cavity 20 to fit within the inner profile of surface 22.

Wall 98 includes a surface 102 that defines a cavity, such as, for example, an arcuate track 104 recessed within surface 102 and configured for moveable disposal of pin 72, as described herein. Arm 100 includes a surface 106 that defines a cavity, such as, for example, an arcuate track 108 recessed within surface 106 and configured for moveable disposal of pin 78, as described herein. With pins 72, 78 fixed within openings 70, 76, saddle 90 is translatable relative to crown 60 along an arcuate pathway, as described herein, such that tracks 104, 106 translate or slide relative to pins 72, 78. In some embodiments, this configuration allows saddle 90 to rotate relative to crown 60 in a plane, such as, for example, a sagittal plane of a body and/or vertebrae.

Saddle 90 includes a surface 109 configured for slidable engagement with tracks 82, 84. Surface 109 extends between ends 92, 94 and is configured for slidable engagement with surface 80 of crown 60 along an arcuate pathway of the components. Surface 110 is configured to engage at least a portion of an implant, such as, for example, a spinal rod (not shown) and is moveable relative to crown 60 in a plane, such as, for example, a sagittal plane of a body and/or vertebrae. Surface 110 defines a concave surface 112 that defines an implant cavity 114. Cavity 20 includes cavity 114.

Receiver 14 defines an axis X2 oriented transverse to axis X1. Saddle 90 is configured to receive and movably support the spinal rod such that the implant can translate axially, rotate and/or pivot relative to receiver 14 along and about axis X2 prior to fixation with saddle 90. In some embodiments, the implant may be disposed within cavity 20 for relative movement in orientations relative to axis X2, such as, for example, transverse, perpendicular and/or other angular orientations such as acute or obtuse, co-axial and/or may be offset or staggered. In some embodiments, axis X2 may be disposed at angular orientations relative to axis X1, such as, for example, acute or obtuse.

In some embodiments, saddle 90 may be elastic and pliable in a configuration to react to forces applied and/or force changes, such as, for example, positioning treatment, patient growth, trauma and degeneration, and/or component creep, deformation, damage and degeneration, to maintain the applied force transmitted from an implant positioned in cavity 20 substantially constant. In some embodiments, saddle 90 can facilitate maintenance of a holding force on an implant positioned in cavity 20 to remain the holding force relatively constant despite growth and changes.

Saddle 90 translates relative to crown 60 via reative slidable translation of tracks 104, 106 relative to pins 72, 78, fixed with crown 60, and along tracks 82, 84 such that saddle 90 is rotatable relative to crown 60 in a plane, such as, for example, a sagittal plane of a body and/or vertebrae. Saddle 90 is rotatable about axis X2 through an angular range o, as shown in FIG. 11. Saddle 90 is pivotable along the arcuate path with crown 60 through cavity 20 in slidable engagement with surface 80 through range α at +/− an angle α1 relative to axis X1. In some embodiments, angular range α may include a range of approximately 0 to 30 degrees. In some embodiments, saddle 90 is disposed with selected receiver 14 for relative movement of an implant in orientations relative to axis X2, such as, for example, transverse, perpendicular and/or other angular orientations such as acute or obtuse, co-axial and/or may be offset or staggered. In some embodiments, saddle 90 moves relative to crown 60 in alternate planes relative to a body, such as, for example, vertical, horizontal, diagonal, transverse, coronal and/or sagittal planes of a body.

Shaft 120 is configured to penetrate tissue, such as, for example, bone. Head 122 is interchangeably engageable with any of the plurality of receivers 14. Head 122 includes a substantially spherical proximal portion configured for moveable disposal with the selected receiver 14 and crown 60. Head 122 includes a surface 124 that defines a plurality of ridges 126 to improve purchase of head 122 with crown 60. An engagement portion of crown 60 is concave or semi-spherical to accommodate the substantially spherical configuration of head 122 such that head 122 is rotatable relative to receiver 14. In some embodiments, this configuration allows shaft 120 to be rotatable relative to axis X1 through a single plane, such as, for example, a transverse plane.

Surface 124 includes interchangeable mating surfaces, such as, for example, arcuate portions 128a and planar portions, such as, for example, flats 128b that are configured for disposal with surface 30 of any of the plurality of receivers 14. Head 122 interfaces with surface 30 such that shaft 120 is rotatable within a transverse plane. In some embodiments, head 122 may be disposed with receiver 14 in alternate fixation configurations, such as, for example, friction fit, pressure fit, locking protrusion/recess, locking keyway and/or adhesive.

Head 122 includes a socket 130 having a hexalobe geometry configured for disposal of a similarly shaped bit of a tool, such as, for example, a driver (not shown) to engage the driver with head 122 to rotate shaft 120. Socket 130 is in communication with cavity 20 such that a driver may be inserted between arms 16, 18 and translated axially, until the bit of the driver is disposed in socket 130. In some embodiments, socket 130 has a cruciform, phillips, square, hexagonal, polygonal, star cross sectional configuration configured for disposal of a correspondingly shaped portion of a driver.

In assembly, operation and use, spinal implant system 10, similar to the systems and methods described herein, includes a selected bone screw 12, which comprises a selected receiver 14 for connection with interchangeable shaft 120 having a selected movement, and is employed with a surgical procedure for treatment of a spinal disorder affecting a section of a spine (not shown) of a patient, as discussed herein. Spinal implant system 10 is employed with a surgical procedure for treatment of a condition or injury of an affected section of the spine. In some embodiments, a selected bone screw 12 comprises a selected receiver 14 for connection with a compatible shaft 120.

The components of spinal implant system 10 include a spinal implant kit, which comprises the plurality of receivers and interchangeable shafts 120. In some embodiments, spinal implant system 10 includes a spinal implant kit, which comprises the plurality of receivers and compatible shafts 120. The plurality of receivers include receivers 14 and alternate receivers, such as those described herein, that interface with interchangeable shafts 120 to comprise one or more bone screw configurations. Selected bone screws 12 and one or a plurality of spinal implants, such as, for example, vertebral rods can be delivered or implanted as a pre-assembled device or can be assembled in situ. The components of spinal implant system 10 may be may be completely or partially revised, removed or replaced.

In one embodiment, a receiver 14 is selected from the kit of the plurality of receivers 14 for interchangeable connection with shaft 120 to comprise a bone screw 12. Ring 36 is disposed with head 122, as described herein. Crown 60 is disposed with the selected receiver 14. Saddle 90 is disposed with crown 60, as described herein, such that surface 110 is positioned with crown 60 and surface 112 is positioned in cavity 20 to receive the spinal rod.

In some embodiments, selected receiver 14, with crown 60 and saddle 90 disposed therein, is engaged with head 122 causing ring 36 to translate, expand and engage groove 34 of the selected receiver 14 such that head 122 translates through ring 36 and is assembled with receiver 14. In some embodiments, head 122 may be assembled with receiver 14 and ring 36 assembled with groove 34. Arcuate portions 128a and flats 128b are disposed with surface 30, as described herein, such that shaft 120 is moveable within a transverse plane. Receiver 14 is attached with a shaft 120 such that receiver 14 is selectively and rotatable relative to shaft 120 within the transverse plane of vertebrae.

Tracks 82, 84, 104, 108 are configured to facilitate translation of saddle 90 relative to crown 60, as described herein. Arms 64, 66 are configured to guide saddle 90 along tracks 82, 84 relative to crown 60. In some embodiments, saddle 90 is selectively translatable along tracks 82, 84, 104, 108 and the arcuate path of saddle 90 relative to crown 60, as described herein, in the sagittal plane to accommodate sagittal anatomical differences. Saddle 90 receives and movably supports the spinal rod such that the spinal rod is movable within cavity 20, as described herein. In some embodiments, this configuration provides movement of saddle 90 to facilitate sagittal accommodation of the spinal rod such that bone screw 12 provides angular accommodation in a transverse plane and a sagittal plane of vertebrae.

In use, for treatment of a spinal disorder, shaft 120 can be threaded and engaged with tissue. In some embodiments, the selected bone screw 12 is disposed adjacent vertebrae at a surgical site and is manipulated to drive, torque, insert or otherwise connect bone screw 12 with vertebrae.

In some embodiments, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the fixation elements with vertebrae. In some embodiments the agent may be hydroxyapatite coating. In some embodiments, the agent may include one or a plurality of therapeutic agents and/or pharmacological agents for release, including sustained release, to treat, for example, pain, inflammation and degeneration.

In some embodiments, the use of microsurgical and image guided technologies may be employed to access, view and repair spinal deterioration or damage, with the aid of spinal implant system 10. The components of spinal implant system 10 can be made of radiolucent materials such as polymers. Radiomarkers may be included for identification under x-ray, fluoroscopy, CT or other imaging techniques.

In some embodiments, spinal implant system 10 can include one or a plurality of bone screws 12 such as those described herein and/or fixation elements, which may be employed with a single vertebral level or a plurality of vertebral levels. In some embodiments, bone screws 12 may be engaged with vertebrae in various orientations, such as, for example, series, parallel, offset, staggered and/or alternate vertebral levels. In some embodiments, bone screws 12 may be configured as multi-axial screws, sagittal angulation screws, pedicle screws, mono-axial screws, uni-planar screws, fixed screws, anchors, tissue penetrating screws, conventional screws, expanding screws. In some embodiments, bone screws 12 may be employed with wedges, anchors, buttons, clips, snaps, friction fittings, compressive fittings, expanding rivets, staples, nails, adhesives, posts, connectors, fixation plates and/or posts.

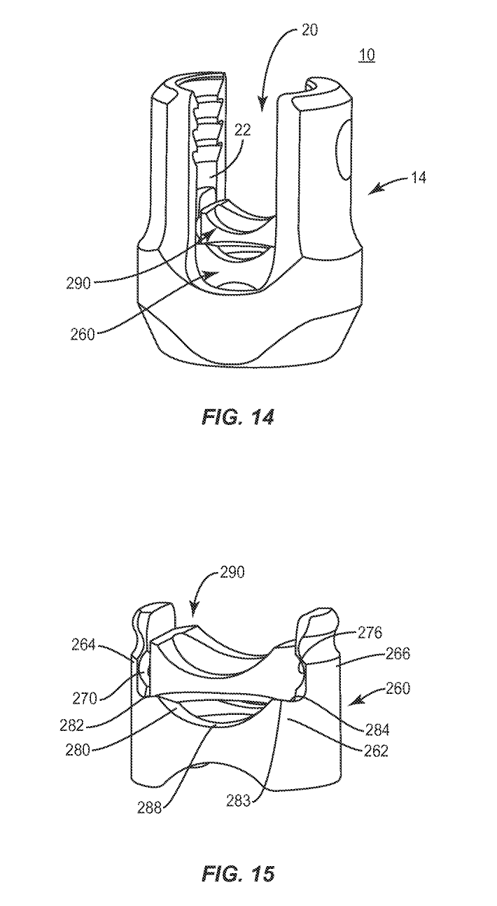

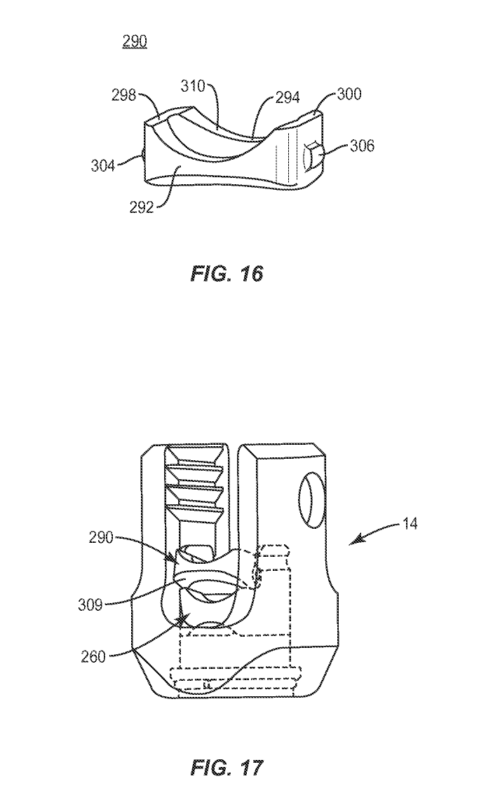

In one embodiment, as shown in FIGS. 14-17, spinal implant system 10, similar to the systems and methods described herein, includes a selected receiver 14, as described herein, having a crown 260 and a saddle 290, and being connectable with shaft 120 (FIG. 5), as described herein. Crown 260, similar to crown 60 described herein, is configured for disposal within the inner profile of surface 22 and/or the perimeter of cavity 20 of the selected receiver 14. Crown 260 includes an outer profile and/or perimeter that fits within the inner profile of surface 22.

Crown 260 includes a wall 262 defining an extension, such as, for example, an arm 264 and an extension, such as, for example, an arm 266. Arms 264, 266 are configured to support relative movement of a part, such as, for example, saddle 290. Wall 262 includes a surface 280 that defines a track 282 adjacent arm 264. Wall 262 includes a surface 283 that defines a track 284 adjacent arm 266. Tracks 282, 284 are configured to facilitate translation of saddle 290 relative to crown 260, as described herein. Arms 264, 266 are configured to guide saddle 290 along tracks 282, 284 relative to crown 260. Wall 262 defines an arcuate portion 288 configured for disposal of at least a portion of an implant, such as, for example, a spinal rod (not shown), which may be positioned with fastener 12 and/or vertebral tissue.

Saddle 290 extends between an end 292 and an end 294. Saddle 290 defines a wall 298 and a wall 300. Walls 298, 300 are configured to fit within the outer profile and/or perimeter of crown 260. In some embodiments, saddle 290 fits within the outer profile and/or perimeter of crown 260 such that the sub-assembly of crown 260/saddle 290 is disposed, such as, for example, inserted and/or loaded upwardly through a lower opening of receiver 14 that communicates with cavity 20 to fit within the inner profile of surface 22.

Saddle 290 includes a surface 309 configured for slidable engagement with tracks 282, 284. Surface 309 extends between ends 292, 294 and is configured for slidable engagement with surface 280 of crown 260 along an arcuate pathway, as described herein, of the components. A surface 310 is configured to engage at least a portion of an implant, such as, for example, a spinal rod (not shown) and is moveable relative to crown 260 in a plane, such as, for example, a sagittal plane of a body and/or vertebrae.

Arm 264 defines a cavity, such as, for example, an arcuate track 270 recessed within arm 264 and configured for moveable disposal of a protrusion 304 of crown 290. Arm 266 defines a cavity, such as, for example, an arcuate track 276 recessed within arm 266 and configured for moveable disposal of a protrusion 306 of crown 290. With saddle 290 assembled with crown 260 and protrusions 304, 306 disposed with tracks 270, 276, saddle 290 is translatable relative to crown 260 along an arcuate pathway, as described herein, such that protrusions 304, 306 translate and/or slide within tracks 270, 276. In some embodiments, this configuration allows saddle 290 to rotate relative to crown 260 in a plane, such as, for example, a sagittal plane of a body and/or vertebra. In some embodiments, protrusions 304, 306 are configured to facilitate connection of saddle 290 with crown 260 and relative motion thereof. In some embodiments, protrusions 304, 306 engage end portions of tracks 270, 276 to define translation, rotation and/or pivot limits of saddle 290 relative to crown 260. In some embodiments, protrusions 304, 306 mate with channels, similar to the tracks described herein, to facilitate relative slidable movement of the components.

Saddle 290 translates relative to crown 260 via relative slidable translation of protrusions 304, 306 within tracks 270, 276 and along tracks 282, 284 such that saddle 290 is rotatable relative to crown 260 in a plane, such as, for example, a sagittal plane of a body and/or vertebrae, similar to crown 60/saddle 90 described herein.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. A spinal implant system comprising:
a plurality of alternate first members including at least one first member comprising an inner surface defining an implant cavity, the at least one first member including a first part being non-rotatable relative to the inner surface and a second part, the first part comprising pins that are positioned within tracks of the second part such that the second part is movable relative to the first part; and
a second member being configured to penetrate tissue and including a mating element engageable with a first member such that the second member is interchangeable with the plurality of first members,
wherein the at least one first member is rotatable relative to the second member in a first plane of a body and the second part defines a portion of the implant cavity and is movable relative to the first part in a second plane of the body.

2. A spinal implant system as recited in claim 1, wherein the mating element interfaces with the at least one first member such that the second member is rotatable about a single axis in the first plane relative to the at least one first member.

3. A spinal implant system as recited in claim 1, wherein the first plane is a transverse plane of the body and the second plane is a sagittal plane of the body.

4. A spinal implant system as recited in claim 1, wherein the first part includes a first surface engageable with the inner surface and a second concave surface configured for disposal of the second part.

5. A spinal implant system as recited in claim 1, wherein the second part is disposed within an outer profile of the first part.

6. A spinal implant system as recited in claim 1, wherein the second part is connected with the first part via at least one protrusion.

7. A spinal implant system as recited in claim 1, wherein the first part includes a pair of arms that support relative movement of the second part.

8. A spinal implant system as recited in claim 1, wherein the first part defines at least one track, the second part being connected to the first part via a protrusion disposed within the track to facilitate movement of the second part relative to the first part.

9. A spinal implant system as recited in claim 8, wherein the at least one track defines an arcuate path.

10. A spinal implant system as recited in claim 1, wherein the second part translates relative to the first part along an arcuate path.

11. A spinal implant system as recited in claim 1, wherein the second part is selectively rotatable to an angular orientation in a range of approximately 0 to 30 degrees relative to the at least one first member within the second plane.

12. A spinal implant system as recited in claim 1, wherein the mating element includes flats engageable with the inner surface to resist and/or prevent rotation of the second member relative to the at least one first member.

13. A spinal implant system as recited in claim 1, wherein the inner surface includes flats and the mating element includes flats that interface in a keyed connection such that the second member pivots through only the first plane relative to the at least one first member.

14. A spinal implant system as recited in claim 1, wherein the inner surface defines a groove configured for disposal of a circumferential ring that defines a gap, wherein the gap defines a slot thickness that is less than a height and a width of the ring.

15. A spinal implant system as recited in claim 1, further comprising a spinal rod disposable with the implant cavity.

16. A spinal implant system comprising:
a plurality of alternate receivers including at least one receiver comprising an inner surface defining an implant cavity, the at least one receiver including a crown fixed in rotation with the inner surface and a saddle, the crown comprising pins that are disposed within arcuate tracks of the saddle such that the saddle is pivotable relative to the crown; and
a bone screw shaft including a head having a mating element engageable with a receiver such that the bone screw shaft is interchangeable with the plurality of receivers,
wherein the at least one receiver is rotatable relative to the bone screw shaft in a transverse plane of a body and the saddle pivots relative to the crown in a sagittal plane of the body.

17. A spinal implant system as recited in claim 16, wherein the saddle is disposed within an outer profile of the crown.

18. A spinal implant system as recited in claim 16, wherein the saddle is selectively rotatable to an angular orientation in a range of approximately 0 to 30 degrees relative to the at least one receiver within the sagittal plane.

19. A spinal implant system as recited in claim 16, wherein the mating element includes flats engageable with the inner surface to resist and/or prevent rotation of the head relative to the at least one receiver.

20. A spinal implant system comprising:
a plurality of alternate implant receivers including at least one implant receiver comprising an inner surface defining an implant cavity, the at least one implant receiver including a crown being non-rotatable relative to the inner surface and a saddle, the crown comprising pins that are positioned within tracks of the saddle such that the saddle is movable relative to the crown; and
a bone screw shaft including a head engageable with an implant receiver such that the shaft is compatible with the plurality of implant receivers,
wherein the at least one implant receiver is selected for connection with the shaft to comprise a bone fastener having rotation of the shaft in a transverse plane and rotation of the saddle in a sagittal plane.
